(19) United States
(12) Patent Application Publication
(10) Pub. No.: US 2002/0137983 A1
(43) Pub. Date: Sep. 26, 2002

(54) Penis erection assistant ring

(76) Inventor: Ling Chang Chen, Nan-Tou Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/813,849

(22) Filed: Mar. 22, 2001

(51) Int. Cl. A61F 5/00

(52) U.S. Cl. 600/41

(57) ABSTRACT

A penis erection assistant ring including a ring body having an outer circumferential face, an inner circumferential face and two end faces. The outer circumferential face has a predetermined conicity. Adjoining sections between the inner and outer circumferential faces and the two end faces are formed with smooth guiding angles. The ring body is composed of two half-rings pivotally connected with each other. One end of each of the half-rings opposite to the pivoted end is provided with a latch structure, whereby the two half-rings are detachably latched with each other to form the ring body.

### BACKGROUND OF THE INVENTION

The present invention is related to an improved penis erection assistant ring composed of two half-rings pivotally connected with each other. A latch structure is provided between the two half-rings, whereby the two half-rings are detachably latched with each other to form the penis erection assistant ring.

Conventionally, the hair circle is used as an assistant ring fitted around the penis of a male to help in erection. The hair circle is muscle tissue of animal and bacteria tend to culture thereon. As a result, the user is subject to infection. Therefore, such hair circle is not sanitary and safe.

Currently, elastic assistant rings made of silicone or rubber have been developed. Such assistant rings can resiliently contract the penis to slow back flow of blood and thus help in erection of the penis. Such assistant rings are more sanitary. However, such assistant rings are subject to scrape to a certain extent due to external force. After a period of use, bacteria may still culture and remain in the scrapes. Furthermore, the silicone or rubber-made assistant rings have stronger resilient force so that the penis is more tightly bound. After erecting, the blood may be unable to circulate. This may result in thrombosis and decay of muscle. On the other hand, after repeatedly used, such assistant rings will suffer resilient failure. Under such circumstance, they will lose original function. Moreover, after repeatedly contracted and expanded, the local scrapes may become apparent fissures or even lead to breakage of the assistant rings. Therefore, such assistant rings are still not sanitary, safe and durable enough.

In addition, such assistant rings entirely encompass the penis so that the ejaculatory duct is tightly bound and the user is often hard to ejaculate.

### SUMMARY OF THE INVENTION

It is therefore a primary object of the present invention to provide an improved penis erection assistant ring composed of two half-rings pivotally connected with each other. One end of each of the half-rings opposite to the pivoted end is provided with a latch structure, whereby the two half-rings are detachably latched with each other to form the ring body. After used, a user can easily stretch open the ring body and remove the ring body from the penis.

It is a further object of the present invention to provide the above penis erection assistant ring in which the inner circumferential face of the ring body is formed with a depression. In use, the depression is aimed at the ejaculatory duct of the penis, whereby a user can normally ejaculate.

The present invention can be best understood through the following description and accompanying drawings wherein:

### BRIEF DESCRIPTION OF THE DRAWINGS

FIG. 1 is a perspective view of the penis erection assistant ring of the present invention;

FIG. 2 is a sectional view taken along line II-II of FIG. 1;

FIG. 3 is a sectional view taken along line III-III of FIG. 1;

FIG. 4 is a side view of the penis erection assistant ring of the present invention, showing that the first half-ring is unlatched from the second half-ring; and

FIG. 5 shows the use of the penis erection assistant ring of the present invention.

### DETAILED DESCRIPTION OF THE PREFERRED EMBODIMENT

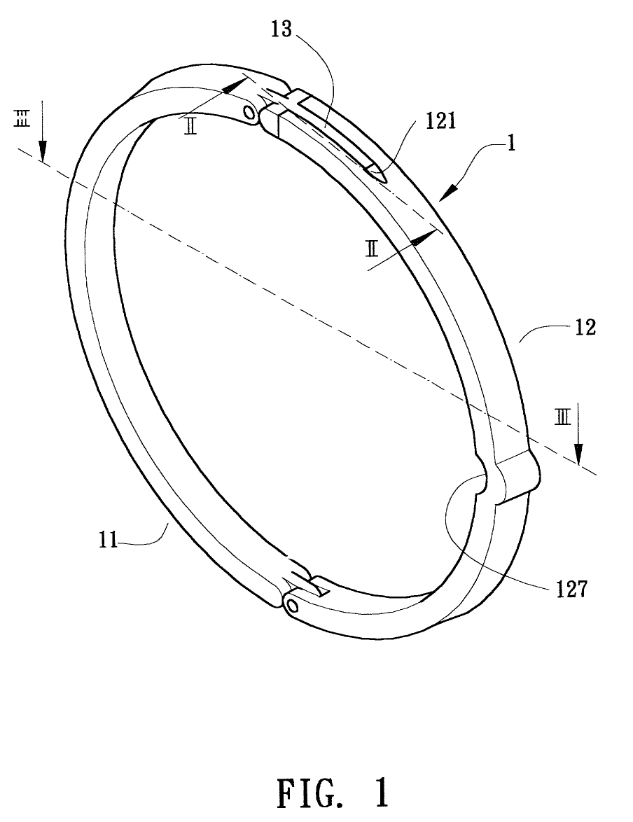

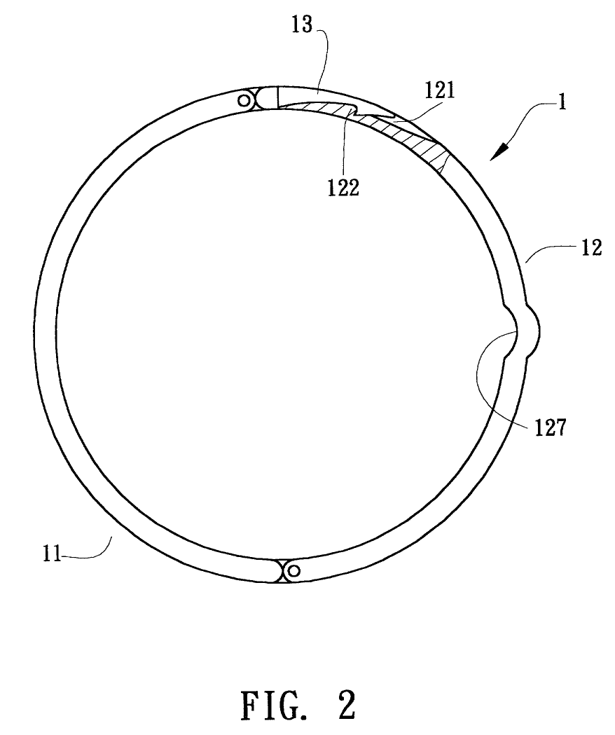

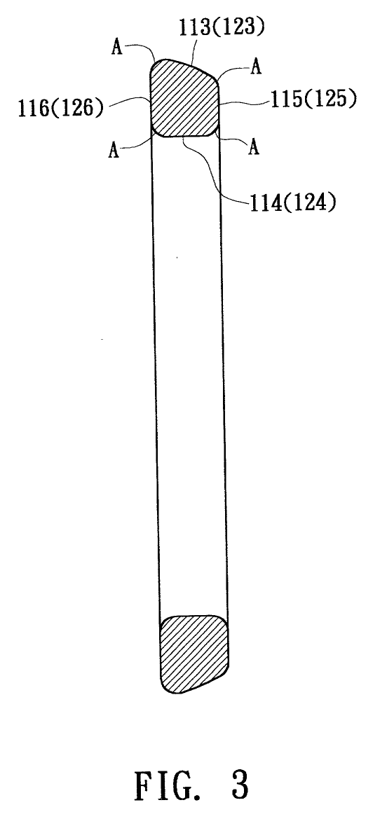

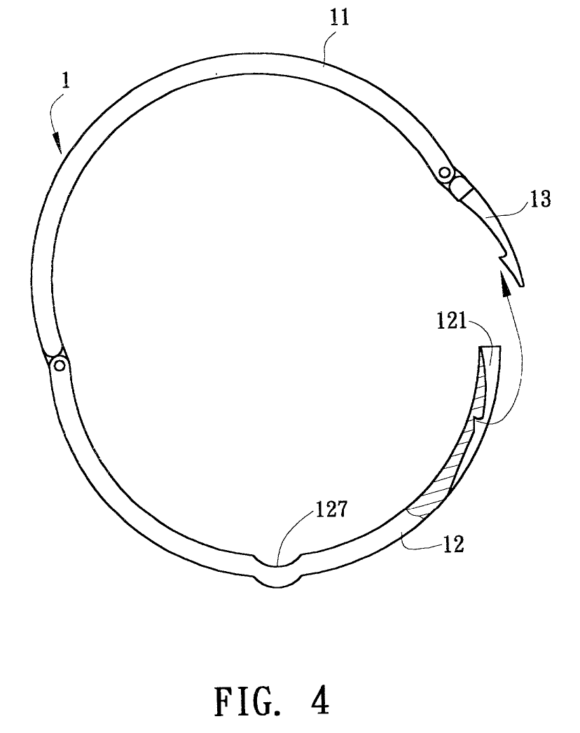

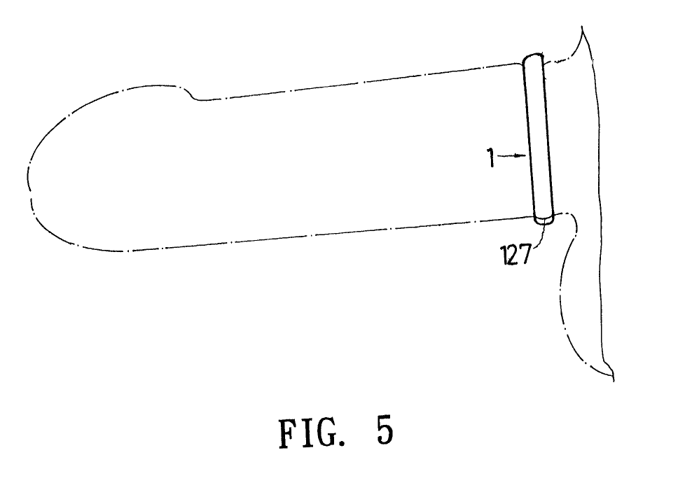

Please refer to FIGS. 1 to 5. The penis erection assistant ring of the present invention includes a ring body 1 having a certain inner diameter and composed of a first half-ring 11 and a second half-ring 12 pivotally connected with each other. One end of the first half-ring 11 opposite to the pivoted end is provided with a hook section 13. One end of the second half-ring 12 opposite to the pivoted end is formed with a recess 121 for inlaying the hook section 13. A projecting block 122 is formed in the recess 121 for retaining the hook section 13 and detachably latching the first half-ring 11 with the second half-ring 12 to form the ring body 1. The ring body 1 has an outer circumferential face 113, 123, an inner circumferential face 114, 124 and two end faces 115, 116, 125, 126. The outer circumferential face 113, 123 has a certain conicity to form a conic face. In addition, adjoining sections between the inner and outer circumferential faces 114, 124, 113, 123 and the two end faces 115, 116, 125, 126 are formed with smooth guiding angle A. Therefore, the ring body 1 has a smooth profile without possibility of scraping a user. In addition, the inner circumferential face 124 of the second half-ring 12 is formed with a depression 127. In use, the depression 127 is aimed at the ejaculatory duct of the penis as shown in FIG. 5, whereby a user can normally ejaculate.

The ring body 1 can have different inner diameters for different users. Furthermore, the ring body 1 is made of metal material which is not subject to oxidation, rusting or scrape so that the sanitation and safety in use can be ensured. In the case that after used, the penis is still erected, the user can pry up the hook section 13 and unlatch the projecting block 122 of the second half-ring 12 from the hook section 13. At this time, the first and second half-rings 11, 12 can stretched open for the user to easily remove the ring body 1 from the penis. Accordingly, the blood can keep circulating and the problems of thrombosis and decay of muscle can be avoided.

The above embodiments are only used to illustrate the present invention, not intended to limit the scope thereof. Many modifications of the above embodiments can be made without departing from the spirit of the present invention.

What is claimed is:

1. A penis erection assistant ring comprising a ring body having a predetermined inner diameter, the ring body further having an outer circumferential face, an inner circumferential face and two end faces, the outer circumferential face having a predetermined conicity to form a conic face, adjoining sections between the inner and outer circumferential faces and the two end faces being formed with smooth guiding angles, said penis erection assistant ring being characterized in that the ring body is composed of two half-rings pivotally connected with each other, one end of each of the half-rings opposite to the pivoted end being provided with a latch structure, whereby the two half-rings are detachably latched with each other to form the ring body.

2. A penis erection assistant ring as claimed in claim 1, wherein the ring body is composed of a first half-ring and a second half-ring pivotally connected with each other, one end of the first half-ring opposite to the pivoted end being provided with a hook section, one end of the second half-ring opposite to the pivoted end being formed with a projecting block for latching the hook section.

3. A penis erection assistant ring as claimed in claim 1, wherein the inner circumferential face of the ring body is formed with a depression.
